(19) United States
(12) Patent Application Publication
(10) Pub. No.: US 2024/0261095 A1
(43) Pub. Date: Aug. 8, 2024

(54) IMPLANT DELIVERY SYSTEM

(71) Applicant: Silara Medtech Inc., Santa Rosa, CA (US)

(72) Inventors: Terry DANIELS, Santa Rosa, CA (US); Mark GEUSEN, Santa Rosa, CA (US); Do UONG, Santa Rosa, CA (US)

(21) Appl. No.: 18/569,191

(22) PCT Filed: Jun. 9, 2022
(86) PCT No.: PCT/US2022/032752

§ 371 Date: Dec. 11, 2023

Related U.S. Application Data
(60) Provisional application No. 63/209,296, filed on Jun. 10, 2021.

(51) Int. Cl. A61F 2/24; A61M 25/09

(52) U.S. Cl. CPC A61F 2/2436; A61F 2/2433; A61M 25/09

(57) ABSTRACT

The present disclosure provides an implant delivery system which is adapted to deliver a cardiovascular prosthetic implant, and comprises: an outer sheath; a balloon and a balloon tube, the balloon being connected to a distal end of the balloon tube; and a nose cone and a guidewire tube, the nose cone being connected to a distal end of the guidewire tube; wherein the balloon tube and the guidewire tube extend longitudinally in the inner cavity of the outer sheath and may pass through the distal end of the outer sheath. With the technical solution provided by the present disclosure, the technical problem that the surgical process is complicated when the balloon is independent of the implant delivery system can be alleviated, and the surgical efficiency can be improved.

### CROSS-REFERENCE TO RELATED APPLICATION

The present disclosure claims the priority of the U.S. Provisional Application No. 63/209,296, filed on Jun. 10, 2021, entitled “IMPLANT DELIVERY SYSTEM, INTRODUCER, HEMODYNAMIC PRESSURE MONITORING SYSTEM AND AIDING DEVICE FOR MONITORING HEMODYNAMIC PRESSURE”, the entire disclosure of which is incorporated herein by reference.

### TECHNICAL FIELD

The present disclosure relates to the technical field of medical instruments, and in particular to an implant delivery system.

### BACKGROUND

In transcatheter valve replacement, a heart valve prosthesis is mounted in a rolled-up state in a tip end portion of a flexible catheter of a delivery system and is advanced through the patient's blood vessel along with the flexible catheter, until the valve prosthesis reaches an implantation position; finally, the heart valve prosthesis at the tip end of the catheter expands to its functional size at the site of a defective native valve.

In the existing heart valve prosthesis implanted via a catheter, there is a heart valve prosthesis with a cuff. In the expansion of this kind of heart valve prosthesis, it is necessary to fill liquid or gas into the cuff to inflate the heart valve prosthesis. After the heart valve prosthesis has been placed at the native valve by the delivery system, due to some physiological and structural reasons, the heart valve prosthesis cannot fully expand or attach well to the heart, such that the implanted heart valve prosthesis cannot meet the clinical requirements in terms of expansion and differential pressure. As a result, it is necessary to introduce a balloon to perform post-expansion reshaping on the heart valve prosthesis. However, the existing balloon is independent of the implant delivery system, which is inconvenient for medical staff to operate, and the surgical process is complicated.

### SUMMARY OF THE INVENTION

The disclosure provides an implant delivery system, in order to address at least the technical problem that the balloon is independent of the implant delivery system, which is inconvenient for medical staff to operate, and the surgical process is complicated.

The above purpose of the present disclosure can be achieved by adopting the following technical solutions.

The invention provides an implant delivery system adapted to deliver a cardiovascular prosthetic implant, the implant delivery system comprising an outer sheath, a balloon, a balloon tube, a nose cone and a guide wire tube. The balloon is connected to a distal end of the balloon tube. The nose cone is connected to a distal end of the guide wire tube. The balloon tube and the guidewire tube are disposed within the outer sheath in a longitudinal extension manner and are configured to be operable to pass through the distal end of the outer sheath.

Furthermore, the guidewire tube is disposed to be at least partially extending through the balloon tube, and the guidewire tube includes a free segment and a bound segment adjacent to the free segment. Wherein the bound segment is defined by a length of the guidewire tube extending longitudinally through the balloon tube, and the free segment is defined by a length of the guidewire tube located outside of the balloon tube and extending longitudinally in parallel with the balloon catheter.

Furthermore, the guidewire tube is configured to be longitudinally movable relative to the balloon tube. Specifically, the balloon tube includes a proximal segment tube, a middle segment tube and a distal segment tube, the middle segment tube is located between the proximal segment tube and the distal segment tube, and the bound segment of the guidewire tube extends longitudinally through the distal segment tube. Preferably, the outer diameter of the middle segment tube of the balloon tube is smaller than that of the proximal segment tube. Preferably, a side wall opening is provided near the proximal end of the distal segment tube of the balloon tube, and the bound segment of the guidewire tube extends longitudinally through the distal segment tube of the balloon tube via the side wall opening. Preferably, the distal segment tube of the balloon tube is configured as a double-lumen tube; the double-lumen tube has a first lumen communicating with the balloon and a second lumen isolated from the balloon; the first lumen of the distal segment tube of the balloon tube is in communication with the lumen of the middle segment tube of the balloon tube; and the bound segment of the guidewire tube extends longitudinally through the second lumen of the distal segment tube of the balloon tube.

Preferably, the cavities of the middle segment tube and the proximal segment tube of the balloon tube are circular in cross section, and the cross section of the cavity of the first lumen of the distal segment tube of the balloon tube is quasi crescent-shaped.

Furthermore, an inner tube isolated from the inner cavity of the balloon is arranged inside the balloon; the second lumen of the distal segment tube of the balloon tube is in communication with the inner tube from the proximal end of the inner tube; the bound segment of the guidewire tube extends longitudinally through the inner tube.

Furthermore, the delivery system further comprises a plurality of positioning wires and an inner sheath adapted to accommodate the plurality of the positioning wires, and the plurality of the positioning wires are configured such that distal ends thereof are connected to an implant. Wherein a proximal end of the inner sheath, a proximal end of the guidewire tube and a proximal end of the balloon tube are configured to extend longitudinally through the outer sheath in parallel and laterally non-nested with each other.

Furthermore, the delivery system further comprises a handle. The proximal end of the outer sheath is connected to the handle, and the handle is configured to drive the outer sheath to move proximally to release the implant.

The features and advantages of the delivery system described above include at least:

The delivery system integrates a balloon and a balloon tube which are disposed within the outer sheath, wherein the balloon may be delivered to the implantation position along with the implant connected to the distal end of the positioning wire. The implant is adjusted using a balloon integrated on the delivery system, which is beneficial for simplifying the surgical process, shortening the surgical operation time and reducing the surgical risk.

The invention provides another implant delivery system, comprising: an outer sheath; a plurality of positioning wires and an inner sheath adapted to accommodate the plurality of the positioning wires, and the plurality of the positioning wires are configured such that distal ends thereof can be connected to an implant; a balloon and a balloon tube, the balloon being connected to a distal end of the balloon tube; and a nose cone and a guidewire tube, the nose cone being connected to a distal end of the guidewire tube. Wherein the inner sheath, the guidewire tube and the balloon tube are disposed within the outer sheath, and the body portions of the inner sheath, the guidewire tube, and the balloon tube are configured to extend longitudinally through the outer sheath in parallel and laterally non-nested with each other. In particular, the inner sheath is separate from the outer sheath, and the material of the inner sheath has a stiffness greater than that of the outer sheath. In particular, the material of the outer sheath is Nylon, and the material of the inner sheath is PEEK.

Furthermore, the inner sheath is a three-lumen tube, and the three-lumen tube includes three independent lumens to be adapted to accommodate three positioning wires. Preferably, an outer profile of the inner sheath is arranged close to the inner peripheral wall of the outer sheath. Specifically, the three lumens of the inner sheath are distributed in an arc shape, and the diameter of a circumcircle of the outer profile of the inner sheath is equal to the inner diameter of the outer sheath.

Furthermore, the delivery system further comprises a handle, and the proximal end of the outer sheath and the proximal end of the inner sheath are connected to the handle. The proximal end of the balloon tube, the proximal end of the guidewire tube and the proximal ends of the plurality of positioning wires all extend beyond the handle. The handle is provided therein with a first seal member which is configured to provide sealing for the inner sheath, the balloon tube and the guidewire tube. Wherein the sealing provided by the first seal member for the balloon tube and the guidewire tube is a longitudinally movable sealing. Preferably, the handle is further provided with a second seal member which is configured to provide a longitudinally movable sealing for the plurality of positioning wires. Specifically, the first seal member may be a sealing gasket, and the second seal member may be a necking member.

The features and advantages of the another implant delivery system described above include at least:

The delivery system integrates a balloon and a balloon tube which are disposed within the outer sheath, wherein the balloon may be delivered to the implantation position along with the implant connected to the distal end of the positioning wire. The position and morphology of the implant are adjusted by the positioning wire and the implant is inflated by delivering the filler to the implant by the positioning wire. The balloon arrives at the implantation position together with the implant to facilitate adjustment of the implant by utilizing the balloon, which is beneficial for simplifying the surgical process and shortening the surgical operation time. The inner sheath, the balloon tube and the guidewire tube are relatively independent in the outer sheath, which can reduce interference to other internal catheters when operating a single internal catheter.

### BRIEF DESCRIPTION OF THE DRAWINGS

In order to more clearly explain the technical solutions in the embodiments of the present disclosure, drawings that need to be used in description of the embodiments will be simply introduced below. Obviously, the drawings in the following description are merely some examples of the present disclosure, and for persons ordinarily skilled in the art, it is also possible to obtain other drawings according to these drawings without making creative efforts.

FIGS. 1A-1B are structural schematics of an implant delivery system of an embodiment;

FIG. 1C is a sectional view taken along the line A-A in FIG. 1A;

FIGS. 2A-2B are schematic views showing the assembling relationship between the balloon tube and the guidewire tube;

FIGS. 3A-3H are schematic views of the balloon tube;

FIGS. 4A-5 are schematic views of the nose cone;

FIGS. 6A-6B are schematic views of a handle;

FIGS. 7 and 8A are schematic views showing the assembling relationship between the carriage and the sealing assembly, the outer sheath, the positioning wires, the balloon tube and the guidewire tube;

FIG. 8B is a cross-sectional view of FIG. 8A with the positioning wires, the balloon tube, and guidewire tube being removed;

FIG. 8C is an exploded schematic view of FIG. 8A with the positioning wires being removed;

FIGS. 9A and 9B are exploded schematic views of the carriage and the sealing assembly;

FIGS. 10A-10H are schematic views showing the operating process of the implant delivery system;

FIG. 11 is a structural schematic of the implant delivery system of another embodiment;

FIGS. 12A-13C are schematic views of the guide sheath and its mating with the outer sheath.

### DETAILED DESCRIPTION OF THE PREFERRED EMBODIMENTS

Hereinafter the technical solutions in the embodiments of the present disclosure will be described clearly and integrally in combination with the accompanying drawings in the embodiments of the present disclosure. Obviously, the described embodiments are merely part of the embodiments of the present disclosure, not all of the embodiments. Any other embodiments obtained by those skilled in the art based on the embodiments of the present disclosure without paying any creative labor fall within the protection scope of the present disclosure.

### The First Embodiment

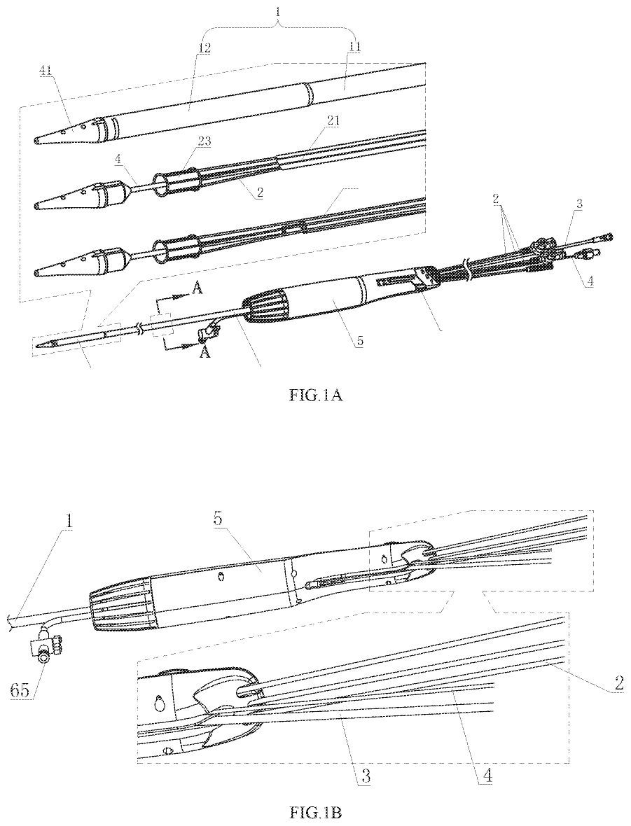

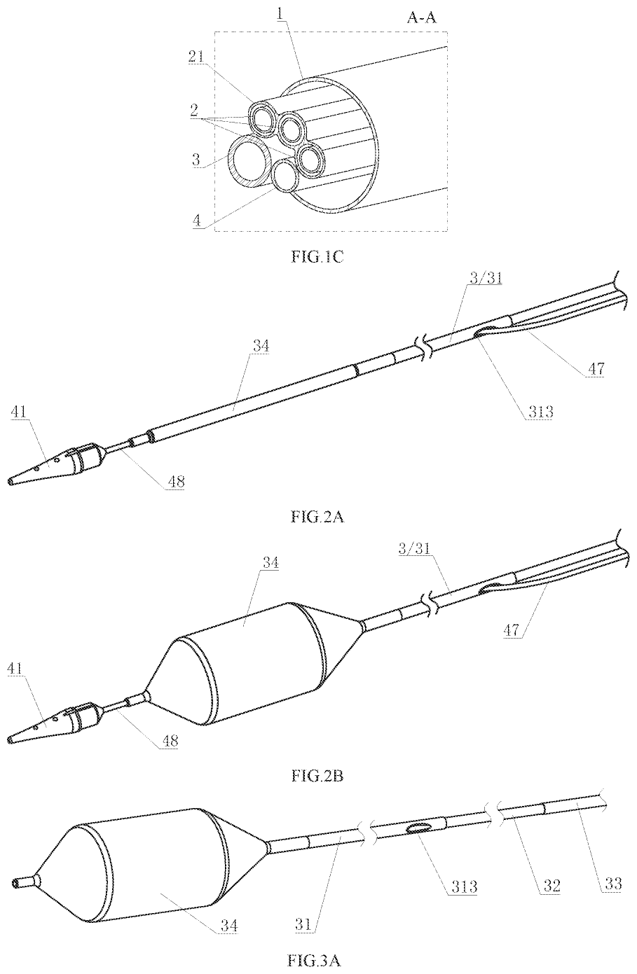

An implant delivery system, as shown in FIGS. 1A, 1B, 2A and 2B, comprises a handle 5, an outer sheath 1, a positioning wire 2, a balloon 34, a balloon tube 3, a guidewire tube 4 and a nose cone 41, wherein a proximal end of the outer sheath 1 is connected to the handle 5. The positioning wire 2 is disposed to pass through the outer sheath 1; a distal end of the positioning wire 2 is configured to be connected to an implant 23, the position of the implant 23 can be adjusted by means of the positioning wire 2 so as to position the implant 23 precisely, and the positioning wire 2 can also provide a fluid passage for filling or recovering medium into or from the implant 23. The balloon 34 is connected to a distal end of the balloon tube 3; the nose cone 41 is connected to a distal end of the guidewire tube 4; the balloon tube 3 and the guidewire tube 4 extend longitudinally in the inner cavity of the outer sheath 1 and can pass through the distal end of the outer sheath 1. The balloon 34 may be provided in the nose cone 41 or in the cavity of the outer sheath 1. The handle 5 is configured to be used to drive the outer sheath 1 to move proximally to release the implant 23. Upon completion of the delivery deployment, the method of disconnecting the distal end of the positioning wire from the implant may include severing an attachment, rotating a screw, withdrawing or shearing a pin, mechanically decoupling interlocked components, electrically separating a fuse joint, removing restricted trapped cylinder from a tube, fracturing an engineering area, removing a collet mechanism to expose a mechanical joint or other methods known in the art.

As shown in FIG. 1A, the outer sheath 1 includes a rod portion 11 and a sheath jacket 12 connected to the distal end of the rod portion 11. The sheath jacket 12 is configured to be adapted for accommodating an inner sheath 21, a balloon tube 3, a guidewire tube 4, an implant 23 and a balloon 34. In some embodiments, the sheath jacket 12 may contain the implant 23 in a contracted state and a portion of the balloon 34 in a contracted state, and the implant 23 and a portion of the balloon 34 are disposed longitudinally separately in the sheath jacket 12 so as to facilitate delivery of the implant 23 and the balloon 23 together to the implantation position. In some embodiments, the inner diameter of the sheath jacket 12 is greater than the inner diameter of the rod portion 11 so that the delivery system can deliver the implant 23 with a larger size. On the other hand, by increasing the inner diameter of the sheath jacket 12, it will be helpful to reduce the loading resistance and the release resistance of the implant 23. The filler such as liquid or gas may be delivered to the implant 23 through the positioning wire 2. The implant 23 is detachably connected to the distal end of the positioning wire 2. Specifically, the implant 23 and the positioning wire 2 are connected by screw thread. For example, the implant 23 is provided with a threaded hole, and the distal end of the positioning wire 2 is provided with a threaded post.

The delivery system integrates the balloon 34 and the balloon tube 3. The balloon 34 may be delivered to the implantation position by the delivery system together with the implant 23 connected to the distal end of the positioning wire 2. The balloon 34 is inflated inside the implant 23 by moving the balloon 34 into the expanded implant 23 via the balloon tube 3 and by delivering the filler to the balloon 34 through the balloon tube 3. The implant 23 is subjected to expansion reshape and shape adjustment, which may cause the implant 23 to fully inflate and achieve good adherence to the physiological structure, this is beneficial for the implanted implant 23 to meet the clinical requirements in expansion and differential pressure, thereby ensuring effective fixation of the implant 23 and minimizing perivalvular leakage, detachment or displacement. The implant 23 is adjusted using a balloon 34 integrated on the delivery system, which is beneficial for simplifying the surgical process, shortening the surgical operation time and reducing the surgical risk. The implant 23 may be a cardiovascular implant, such as a cardiac aortic valve, a mitral valve, a pulmonary valve and/or a tricuspid valve, or the like.

As shown in FIG. 1A, the delivery system further comprises an inner sheath 21 that is disposed to pass through the outer sheath 1, the inner sheath 21 is adapted to accommodate the positioning wire 2, the positioning wire 2 is disposed to pass through the inner sheath 21 and extends out from the distal end of the inner sheath 21, and the positioning wire 2 can move freely longitudinally in the inner sheath 21. The inner sheath 21 provides an independent cavity for the positioning wire 2 to avoid interference by other components located within the outer sheath 1 during the operation of the positioning wire 2. In some embodiments, the inner sheath 21 extends longitudinally in the interior cavity of the outer sheath and passes through the proximal end of the outer sheath 1.

In some embodiments, the inner sheath 21 is separate from the outer sheath 1, and the inner sheath 21 and the outer sheath 1 are made of different materials. For example, the material of the outer sheath is Nylon, and the material of the inner sheath is polyether-ether-ketone (PEEK). The inner sheath 21 made of PEEK material advantageously provides support for the positioning wire 2. In addition to Nylon, the material of the outer sheath may also be a material known in the art that can be used for the outer sheath. In addition to PEEK, the inner sheath may be made of other resin or metal materials that are harder than the outer sheath.

The number of the positioning wire may be 1, 2 or more. In some embodiments (as shown in FIG. 1C), the delivery system comprises three positioning wires 2. Correspondingly, the inner sheath 21 may be a three-lumen tube 21, and the three-lumen tube 21 includes three independent lumens to be adapted to accommodate three positioning wires. The three positioning wires 2 are located respectively in independent lumens, which can avoid mutual contact between the three positioning wires and mutual interference during the movement of the positioning wires. In some embodiments, the outer profile of the three-lumen tube 21 is arranged close to the inner peripheral wall of the outer sheath to make full use of the inner space of the outer sheath, thereby facilitating reduction of the dimension of the outer profile of the outer sheath 1. In a preferred embodiment, as shown in FIG. 1C, the three lumens of the three-lumen tube 21 are distributed in an arc shape, the diameter of an circumcircle of the outer profile of the three-lumen tube 21 is equal to the inner diameter of the outer sheath 1, so as to make full use of the inner space of the outer sheath 1 and reduce the diameter of the outer sheath 1. In the meantime, in the case where the inner diameter dimension of the outer sheath 1 is constant, the arc-shaped design in which the outer profile of the three-lumen tube 21 snugly abuts the inner wall of the outer sheath 1 may reserve more space for the balloon tube 3 and the guidewire tube 4 in the outer sheath 1, such that mutual interference between the balloon tube 3 and the guidewire tube 4 can be reduced when they are operated independently.

In the embodiments shown in FIGS. 1A, 1B and 1C, a longitudinally extending cavity is provided in the guidewire tube 4 for accommodating the guidewire (not shown in the figures). The guidewire tube 4 penetrates at least partially into the handle 5 and into the outer sheath 1 connected to the handle 5. The connection segment 45 (see FIG. 4A) at the proximal end of the nose cone 41 is detachably connected to the distal end of sheath jacket 12 to facilitate separation of the nose cone 41 from the sheath jacket 12 upon release of the implant out of the sheath jacket. In a preferred embodiment, the outer diameter of the connection segment 45 of the nose cone 41 is in an interference fit with the inner diameter of the sheath jacket 12, which can effectively reduce a fish mouth (a gap between the distal end portion of the sheath jacket 12 and the nose cone 41 when the nose cone 41 is in the bending direction) formed by the sheath jacket 12 when the nose cone 41 is bent. The distal end conical portion of the nose cone 41 extends beyond the outer sheath 1. The distal end portion of the guidewire tube 4 is connected to the proximal end of the nose cone 41. The guidewire tube 4 may be bonded to the proximal end portion of the nose cone 41, and the interior cavity of the guidewire tube 4 communicates with the shaft hole of the nose cone 41. In some embodiments, the distal end portion of the guidewire tube 4 is inserted into the proximal shaft hole of the nose cone 41.

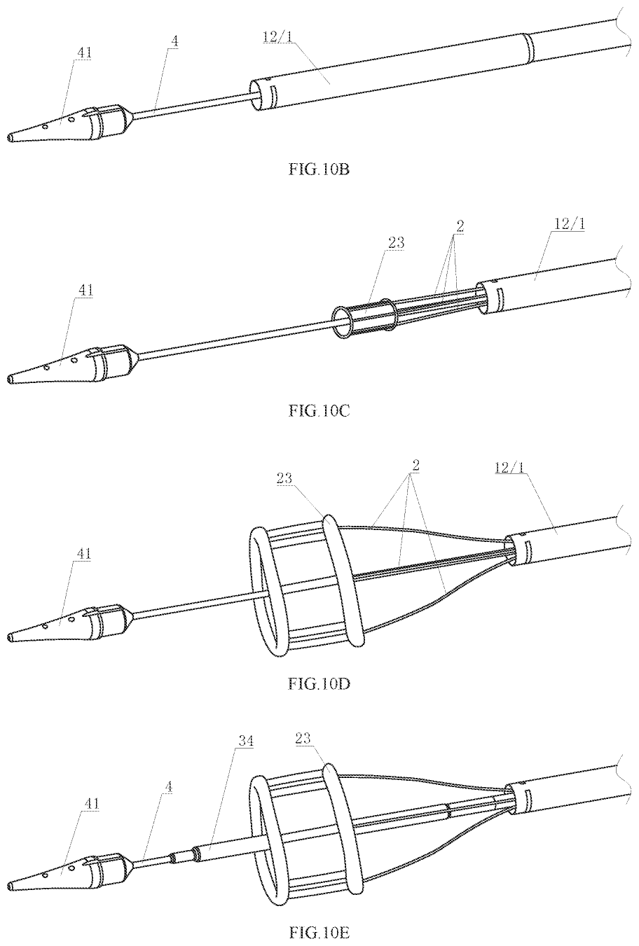

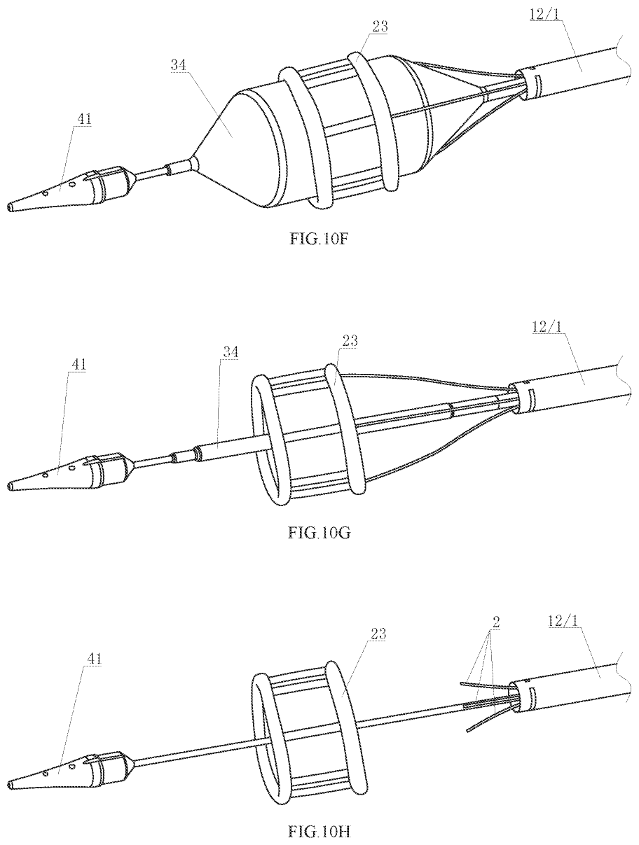

Referring to FIGS. 10A to 10H, after the implant 23 is delivered to the target position, the longitudinal position of the balloon 34 is adjusted so that the balloon 34 passes through and is laterally aligned with the implant 23. The balloon 34 is expanded and inflated to reshape the expansion of the implant 23 for post-expansion. Depending on the surgical requirements, the balloon 34 may be firstly adjusted so as to match the primary tissue before the implant 23 is brought into a match with the primary tissue after it is delivered to the target position, and the balloon 34 is expanded and inflated to adjust the morphology of the primary tissue to achieve pre-expansion and facilitate subsequent smooth placement of the implant 23.

The balloon 34 may be a semi-compliant balloon, the nominal pressure thereof is about 1˜2 ATMs, and the bursting pressure is about 3 ATM. The nominal pressure and the bursting pressure of the balloon 34 may be adjusted as desired. The material of the balloon 34 may be polyethylene (PE), polyurethane or nylon. After the native valve is pre-expanded, the expanded heart valve prosthesis implant 23 is positioned at the native valve. The balloon 34 with the pressure of 1˜3 ATMs may assist in the post-expansion of the expanded prosthesis again.

In some embodiments, the balloon tube 3 and the guidewire tube 4 are arranged side by side in the outer sheath 1. The guidewire tube 4 is longitudinally movable relative to the balloon tube 3, through which the position of the balloon 34 can be independently adjusted.

In some other embodiments, as shown in FIG. 2B, the guidewire tube 4 is disposed to partially pass through the balloon tube 3, and the distal end of the guidewire tube 4 extends beyond the distal end of the balloon 34. The guidewire tube 4 includes a free segment 47 and a bound segment 48 adjacent to the free segment 47; wherein the free segment 47 is defined by a length of the guidewire tube 4 from its proximal end portion to a location where the guidewire tube 4 penetrates into the balloon tube 1. The free segment 47 of the guidewire tube 4 and the balloon tube 3 extend longitudinally in parallel in the outer sheath 1. The bound segment 48 is defined by a length of the guidewire tube 4 from a location where it penetrates into the balloon tube 3 to the distal end portion of the guidewire tube 4. That is, the free segment 47 of the guidewire tube 4 is defined by a length of the guidewire tube 4 located outside of the balloon tube 3 and extending longitudinally in parallel with the balloon catheter 3, and the bound segment 48 is defined by another length of the guidewire tube 4 which is disposed to pass through the balloon tube 3 and extends longitudinally distally. In a preferred embodiment, the guidewire tube 4 is longitudinally movable relative to the balloon tube 3. When the guidewire tube 4 moves distally relative to the balloon tube 3, the bound segment 48 becomes longer and the free segment 47 becomes shorter. When the guidewire tube 4 moves proximally relative to the balloon tube 3, the bound segment 48 becomes shorter and the free segment 47 becomes longer.

A part of the guidewire tube 4 is disposed to pass through the balloon tube 3. When the position of the balloon 34 is adjusted by the balloon tube 3, the balloon 34 moves along the guidewire tube 4, through which the balloon 34 can be guided, so as to facilitate more easily and quickly moving the balloon 34 into the implant 23. The free segment 47 of the guidewire tube 4 is independent of the balloon tube 3, facilitating independent manipulation of the guidewire tube 4 and the balloon tube 3. For example, in some embodiments, prior to releasing the implant 23, it is necessary to move the nose cone 41 distally with respect to the outer sheath 1 firstly through the guidewire tube 4. At this time, the balloon tube 3 is held stationary with respect to the outer sheath 1. When the implant 23 is precisely positioned at the proper implantation position, it is necessary to move the balloon 34 by the balloon tube 3 to expand and reshape the implant 23. At this time, the guidewire tube 4 keeps stationary.

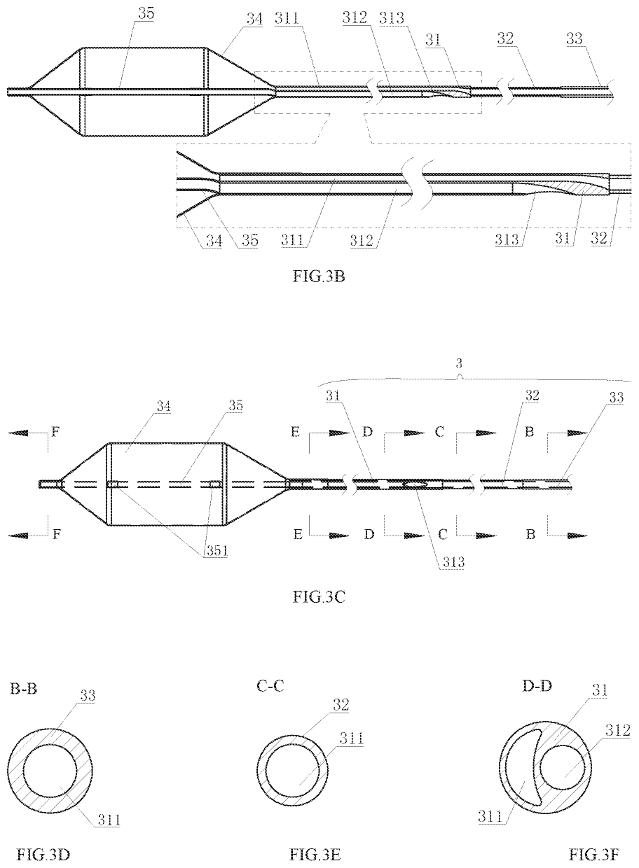

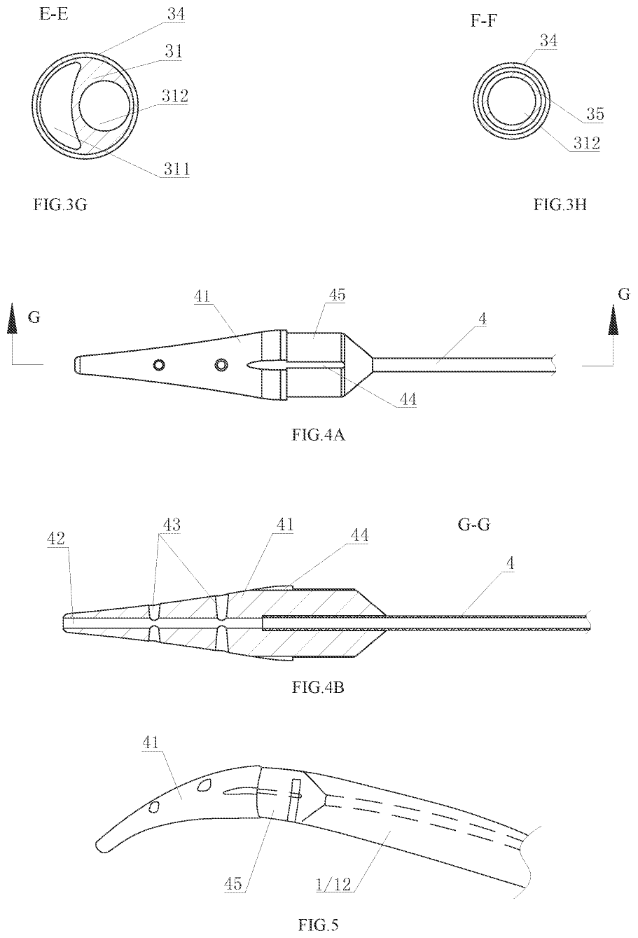

In some embodiments, as shown in FIGS. 2A-3C, the balloon tube 3 includes a proximal segment tube 33, a middle segment tube 32, and a distal segment tube 31. The middle segment tube 32 is located between the proximal segment tube 33 and the distal segment tube 31. The proximal segment tube 33, the middle segment tube 32, and the distal segment tube 31 are sequentially connected and communicated with each other. The bound segment 48 of the guidewire tube 4 extends longitudinally through the distal segment tube 31. The outer diameter of the middle segment tube is smaller than that of the proximal segment tube, and the inner diameter of the middle segment tube is equal to that of the proximal segment tube. The outer diameter of the middle segment tube 32 is small and the tube wall is thin, so that the middle segment of the balloon tube 3 is more flexible, which will facilitate the bending and stecring of the balloon tube 3. The outer diameter of the proximal segment tube 33 is large and the tube wall is thick, which can provide more support for the balloon tube 3 and facilitate the proximal operator to manipulate the balloon tube 3. In addition, the middle segment tube 32 having a smaller outer diameter is advantageous in saving the space occupied by the balloon tube 3 in the outer sheath 1. The space-saving with the middle segment tube 32 will further facilitate the independent manipulation of the balloon tube 3 and the inner sheath 21. In summary, the distal and proximal segments of the balloon tube 3 are stiffer than the middle segment, which can provide a strong supporting force, while the middle segment is relatively flexible, and the cooperation of these features will facilitate the independent manipulation of the balloon tube 3, the guidewire tube 4 and the inner sheath 21 by the operator.

The guidewire tube 4 may enter the distal segment tube 31 from the proximal end face of the distal segment tube 31, or it may enter the distal segment tube 31 from a proximal side wall of the distal segment tube 31. In the embodiment shown in FIG. 2B, a side wall opening 313 is provided near the proximal end of the distal segment tube 31, and the guidewire tube 4 extends longitudinally through the distal segment tube via the side wall opening 313.

As shown in FIG. 3B, the distal segment tube 31 of the balloon tube 3 is a double-lumen tube 31. The double-lumen tube 31 has a first lumen 311 communicating with the balloon 34 and a second lumen 312 isolated from the balloon 34. The first lumen 311 is in communication with the lumen of the middle segment tube 32, and the bound segment 48 of the guidewire tube 4 extends longitudinally through the second lumen 312. Inside the balloon 34, an inner tube 35 is provided which is isolated from the inner cavity of the balloon 34. The second lumen 312 is in communication with the inner tube 35, and the bound segment 48 of the guidewire tube 4 extends longitudinally through the inner tube 35. The first lumen 311 is used for delivering a filling medium (liquid or gas) to inflate the balloon 34, and the second lumen 312 is used for accommodating the guidewire tube 4. Part of the bound segment 48 of the guidewire tube 4 is disposed to pass through the second lumen 312 and the inner tube 35, and the guidewire tube 4 can move in the second lumen 312 and the inner tube 35 to facilitate adjustment of the position of the balloon 34 while the guidewire tube 4 remains stationary. In some embodiments, as shown in FIGS. 3A and 3G, the cavities of the middle segment tube 32 and the proximal segment tube 33 are circular in cross section. The cross section of the first lumen 311 of the double-lumen tube 31 may be quasi crescent-shaped, and the cross section of the second lumen 312 may be circular. As a preferred embodiment, the cross-sectional area of the first lumen 311, the cross-sectional area of the cavity of the middle segment tube 32, and the cross-sectional area of the cavity of the proximal segment tube 33 are equal, which will further facilitate rapid inflation or evacuation of the balloon 34 on the basis of the aforementioned independently and flexibly adjustable balloon and guidewire tube, avoiding that the working efficiency of the balloon is reduced due to reduction of the cross-sectional area of a certain section.

The manner of engagement between the bound segment 48 of the guidewire tube 4 and the balloon 34 is not limited to the manner described above. In other cases, the guidewire tube 4 passes through and is secured to the balloon 34. The positions of the balloon 34 and the guidewire tube 4 may be adjusted in synchronization so that the balloon 34 is brought into a match with the primary tissue or the implant 23. Specifically, the guidewire tube 4 extends through the balloon 34, the lumen of the guidewire tube 4 is spaced apart from the inner cavity of the balloon 34, and the guidewire tube 4 and the balloon 34 may be integrally formed.

In the embodiments shown in FIGS. 3B to 3H, the balloon 34 is provided therein with an inner tube 35 which is isolated from the balloon 34, and the guidewire tube 4 extends through the inner tube 35. The proximal end of the inner tube 35 is connected to the distal end of the distal segment tube 31, and the distal end of the inner tube 35 may extend beyond the balloon 34, and the balloon 34 is fixed to the inner tube 35. The guidewire tube 4 is movable in the inner tube 35. That is, the balloon tube 3 can drive the balloon 34 to move together longitudinally relative to the guidewire tube 4 to facilitate adjustment of the position of the balloon 34. For example, in some embodiments, the guidewire tube 4 may remain stationary, while the balloon 34 and the balloon tube 3 may move distally. In a preferred embodiment, as shown in FIG. 2B, the inner tube 35 located within the balloon 34 is provided on the central axis of the balloon. In some embodiments, a third marking band 351 is provided on the inner tube 35 to show the position of the balloon 34.

As shown in FIGS. 4A and 4B, the nose cone 41 is provided with a shaft hole 42 communicating with the guidewire tube 4. The shaft hole 42 is configured to allow a guidewire in the guidewire tube 4 to pass therethrough. The guidewire tube 4 and the shaft hole 42 of the nose cone 41 are used to accommodate the guidewire, which can extend from the lumen of the guidewire tube 4 into the shaft hole 42. In some embodiments, the nose cone 41 is provided with a plurality of side holes 43 in communication with the shaft hole 42. In a preferred embodiment, a plurality of side holes 43 are provided in the radial direction of the nose cone 41, and the number of which is an even number, such as 2, 4, and etc. In the case where the shaft hole 42 is blocked by the heart wall, blood flows from the periphery of the nose cone 41 through the plurality of side holes 43 and is kept in flow communication with the blood flow in the nose cone 41, which facilitates accurate measurement of the blood flow pressure at the periphery of the nose cone 41 by a pressure sensor disposed within the nose cone 41.

The distal end portion of the guidewire tube 4 is connected to the proximal end of the nose cone 41, and the interior cavity of the guidewire tube 4 communicates with the shaft hole of the nose cone 41. The guidewire tube 4 may be bonded to the proximal end portion of the nose cone 41. In some embodiments, the distal end portion of the guidewire tube 4 is inserted into the proximal shaft hole of the nose cone 41.

As shown in FIGS. 4A and 5, the connection segment 45 at the proximal end of the nose cone 41 is detachably connected to the distal end of the sheath jacket 12, and the distal end of the nose cone 41 extends beyond the outer sheath 1. In a preferred embodiment, the outer diameter of the connection segment 45 of the nose cone 41 is in an interference fit with the inner diameter of the sheath jacket 12, which can effectively reduce a fish mouth (a gap between the distal end portion of the sheath jacket 12 and the nose cone 41 when the nose cone 41 is in the bending direction) formed by the sheath jacket 12 when the nose cone 41 is bent. In a more preferred embodiment, a connection segment 45 with a larger length, such as greater than 5 mm, may be provided to avoid separation of the nose cone 41 from the sheath jacket 12 due to a larger fish mouth when the nose cone 41 is bent.

In some embodiments, the outer side wall of the nose cone 41 is provided thereon with a flushing groove 44 in communication with the inner cavity of the outer sheath 1. During the flushing of the inner cavity of the outer sheath 1, air and liquid can be smoothly discharged from the flushing groove 44. Specifically, the flushing groove 44 extends longitudinally through the outer side wall of the nose cone 41 along the proximal end portion of the nose cone 41. A plurality of flushing grooves 44 are distributed along the circumferential direction of the nose cone 41 on the side wall of the nose cone 41.

### The Second Embodiment

An implant delivery system, as shown in FIGS. 1C, 8A to 9B, comprises an outer sheath 1, a plurality of positioning wires 2 and an inner sheath 21 adapted to accommodate the plurality of the positioning wires 2, a balloon tube 3 and a balloon 34, and a guidewire tube 4 and a nose cone 41. The plurality of positioning wires are configured such that distal ends thereof are detachably connected to the implant. The balloon 34 is connected to a distal end of the balloon tube 3, and the nose cone 41 is connected to a distal end of the guidewire tube 4. Wherein, the inner sheath 21, the guidewire tube 4, and the balloon tube 3 run through and are disposed in the outer sheath 1, and the body portions of the inner sheath 21, the guidewire tube 4, and the balloon tube 3 are configured to extend longitudinally through the outer sheath 1 in parallel and laterally non-nested with each other. Referring to FIGS. 1C, 2A and 3A together, the body portions of the inner sheath 21, the guidewire tube 4, and the balloon tube 3, in particular, refer to portions corresponding to the portions of the guidewire tube 4 and the balloon tube 3 where no interference has occurred from proximal to distal. In some embodiments, the proximal ends of the inner sheath 21, the balloon tube 3 and the guidewire tube 4 extend beyond the proximal end of the outer sheath 1. The distal ends of the plurality of positioning wires 2 extend through the inner sheath 21, and the proximal ends of the positioning wires 2 extend beyond the proximal end of the inner sheath 21. The distal end of the inner sheath 21 may extend through the outer sheath 1. In a preferred embodiment, the distal end of the inner sheath 21 is always located in the inner cavity of the outer sheath 1, and the inner sheath 21 and the outer sheath 1 are designed separately. According to the embodiment of the present disclosure, the inner sheath 21 is configured a three-lumen tube 21 which includes three independent lumens to be adapted to accommodate three positioning wires.

In a preferred embodiment, an outer profile of the inner sheath 21 is arranged close to the inner peripheral wall of the outer sheath. Specifically, the three lumens of the inner sheath 21 are distributed in an arc shape, so that the diameter of an circumcircle of the outer profile of the inner sheath is equal to the inner diameter of the outer sheath 1. Such arrangement that the inner sheath 21, the guidewire tube 4 and the balloon tube 3 are disposed in the outer sheath 1 makes it possible to minimize the overall profile of balloon-integrated delivery system, on the basis of ensuring the respective independent operability and reducing mutual interference.

The delivery system further comprises a handle 5, and the proximal end of the outer sheath 1 and the proximal end of the inner sleeve 21 are connected to the handle 5. The proximal ends of the balloon tube 3, of the guidewire tube 4 and of the positioning wires 2 all extend beyond the handle 5. The handle is provided with a first seal member which is configured to provide sealing for the inner sheath 21, the balloon tube 3 and the guidewire tube 4, so as to prevent blood from flowing out of the body along the outer surfaces of the inner sheath 21, the balloon tube 3 and the guidewire tube 4 after the delivery system enters the body. Depending on whether there is relative movement between the sealing member and the component to be sealed, the sealing provided by the sealing member can be divided into two categories, a stationary sealing and a movable sealing. Thus, the first seal member may provide either or both of these two types of sealing. In some embodiments, the inner sheath 21 is fixedly connected to the first seal member, and the first seal member provides a stationary sealing for the inner sheath 21. The balloon tube 3 and the guidewire tube 4 are longitudinally movable relative to the first seal member, and the first seal member provides a longitudinally movable sealing for the balloon tube 3 and the guidewire tube 4.

As shown in FIG. 8A, the handle is provided therein with a second seal member which provides sealing for the plurality of positioning wires 2. The second seal member serves to seal the gap between the plurality of positioning wires 2 and the cavity of the inner sheath 21, preventing blood from flowing out of the body along the outer surface of the positioning wire 2 after the delivery system enters the body.

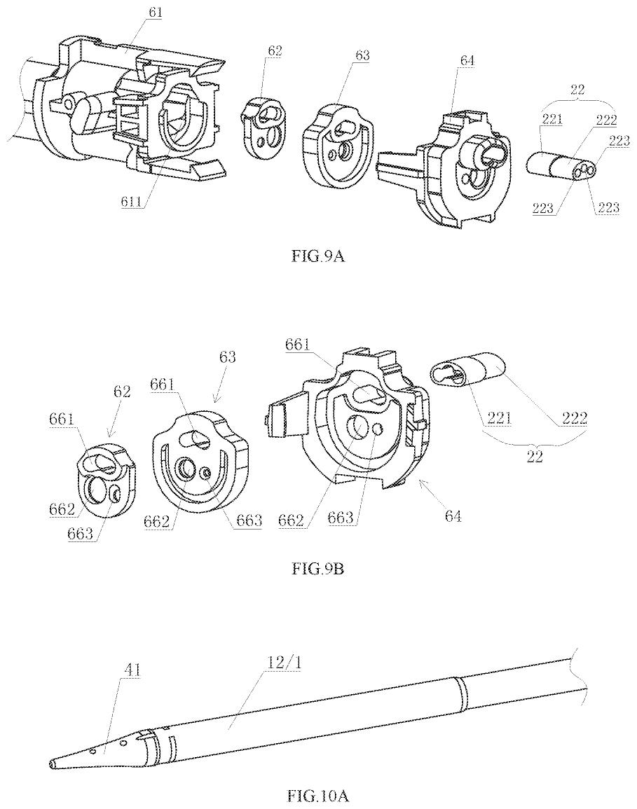

As shown in FIGS. 8B, 8C, and 9B, the first seal member is a sealing gasket 63 which is provided with a first through hole 661, a second through hole 662, and a third through hole 663. The inner sheath 21 passes through and is sealed by the first through hole 661. The balloon tube 3 passes through and is sealed by the second through hole 662, and the guidewire tube 4 passes through and is sealed by the third through hole 663. The second seal member is a necking member 22 through which the positioning wire 2 passes longitudinally, and which is configured to be adapted to provide a longitudinally movable sealing for the positioning wire 2 relative to the inner sheath 21.

As shown in FIG. 1A, a lock clamp 7 is further arranged in the handle, and a plurality of positioning wires 2 pass through the lock clamp 7 which can be used for locking the plurality of positioning wires 2. The implant 23 is connected to the distal end of the positioning wire 2. After the implant 23 is delivered to the implantation position, the positioning wire 2 is locked by using the lock clamp 7, which is favorable for stabilizing the position of the implant 23, therefore, the interference to the positioning wire 2 and the implant 23 when adjusting the balloon tube 3 can be reduced. After the implant 23 is pre-installed in the delivery system, the lock clamp 7 is used to lock the positioning wire 2 during the delivery of the implant 23 to the target position of the human body, so as to be able to prevent relative movement between the outer sheath 1 and the nose cone 41. When the outer sheath 1 moves proximally, the lock clamp 7 can be used to keep the positioning wire 2 to be locked, thereby smoothly releasing the implant 23. If it is necessary to adjust the position of the implant 23, the locking of the positioning wire 2 can be released. Specifically, the specific structure of the lock clamp 7 is not particularly limited here as long as the locking and unlocking functions can be realized.

### The Third Embodiment

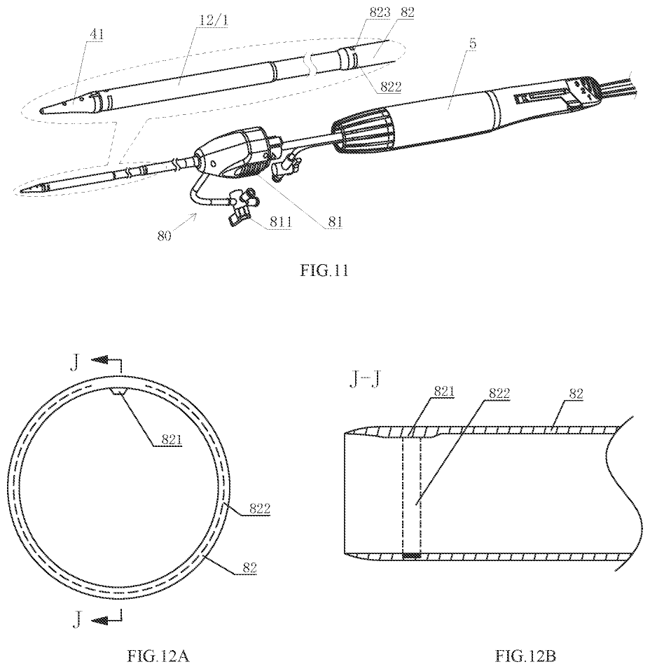

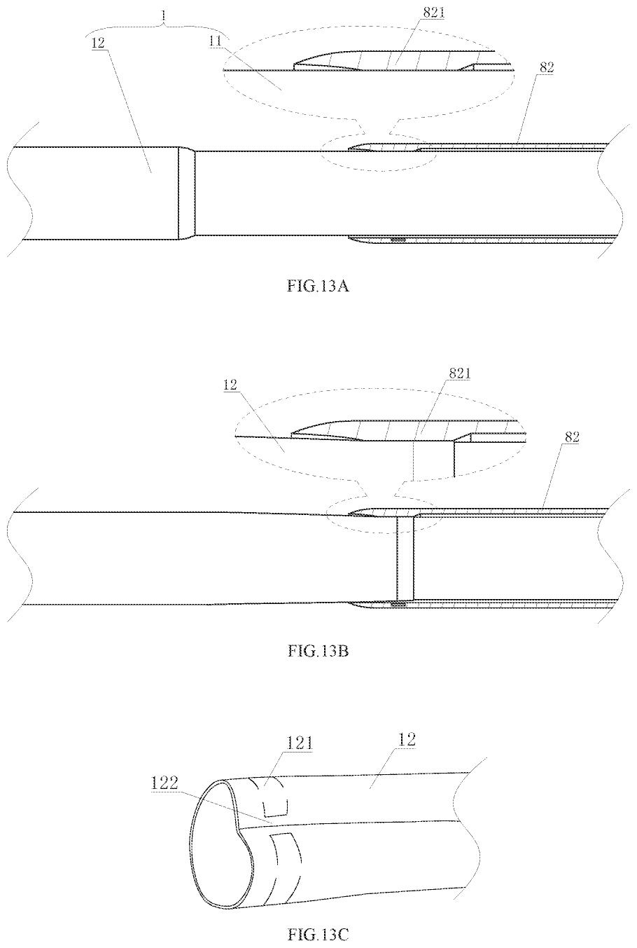

As shown in FIG. 11, an implant delivery system is adapted to deliver a cardiovascular prosthetic implant, the delivery system typically completes delivery of the cardiovascular prosthetic implant in conjunction with the introducer 80. The introducer 80 includes an introducer handle 81 and a guide sheath 82. As shown in FIGS. 12A and 12B, an inner wall of the distal end of the guide sheath 82 is provided with a convex structure 821. The delivery system comprises an outer sheath 1 which passes through the introducer 80 via the guide sheath 82, and the distal end of which extends beyond the distal end of the guide sheath 82. An outer diameter of the distal end of the outer sheath is larger than an inner diameter of the distal end the guide sheath 82. The convex structure 821 serves to reduce the outer profile of the distal end of the outer sheath 1 when the distal end of the outer sheath 1 enters the guide sheath 82 from the distal end of the guide sheath 82.

In some specific embodiments, the outer sheath 1 includes a rod portion 11 and a sheath jacket 12 connected to the distal end of the rod portion 11. The inner diameter of the sheath jacket 12 is greater than the inner diameter of the rod portion 11, and the sheath jacket 12 is adapted to accommodate the cardiovascular prosthetic implant. The rod portion 11 is adapted to extend through the guide sheath 82, and the outer diameter of the sheath jacket 12 is larger than the inner diameter of the guide sheath 82. The sheath jacket 12 is configured to be reduced in the outer profile thereof under the action of the convex structure of the guide sheath 82 as it enters the guide sheath 82 from the distal end of the guide sheath 82.

In some embodiments, the outer diameter of the sheath jacket 12 at the distal end of the outer sheath 1 is greater than the inner diameter of the guide sheath 82, and is about 20 Fr. The outer diameter of the rod portion 11 is smaller than the inner diameter of guide sheath tube 82, and is about 18 Fr. In other embodiments, the sheath jacket 12 has an outer diameter of about 25 Fr. The thickness of the sheath jacket 12 is about 0.2 mm, and for example, the thickness of the sheath jacket 12 is in the range of 0.18 to 0.22 mm, to provide both reliable support for the implant 23 and ease of folding of the sheath jacket 12 as the sheath jacket 12 passes through the guide sheath 82, such that the outer profile of the sheath jacket 12 is reduced, for example, as the sheath jacket 12 is withdrawn from the guide sheath 82.

As shown in FIG. 11 and described above, the outer sheath 1 may be pre-installed into the guide sheath 82 prior to performing the minimally invasive surgery. For example, the manufacturer of the delivery system pre-installs the outer sheath 1 into the guide sheath 82. For example, prior to attaching the handle 5 to the proximal end of the outer sheath 1, the proximal end of the outer sheath 1 extends through the guide sheath 82.

In some embodiments, as shown in FIGS. 12A and 12B, an inner wall of the distal end of the guide sheath 82 is provided with a convex structure 821 which is configured to bend inward the distal side wall of the outer sheath 1. As shown in FIGS. 13A to 13C, the outer diameter of the sheath jacket 12 at the distal end of the outer sheath 1 is greater than the inner diameter of the guide sheath 82, and the outer diameter of the rod portion 11 of the outer sheath 1 is smaller than the inner diameter of guide sheath 82. When the sheath jacket 12 is withdrawn from the guide sheath 82, i.e., when the sheath jacket 12 enters the guide sheath 82 from the distal end of the guide sheath 82 and the sheath jacket 12 abuts against the convex structure 821, the sheath jacket 12 forms a crease that is folded radially inward in the lengthwise direction to reduce the outer circumferential dimension of the sheath jacket 12, such that the outer profile of the sheath jacket 12 is reduced to facilitate withdrawal of the sheath jacket 12 from the guide sheath 82. Specifically, the convex structure 821 extends in the longitudinal direction, and the number thereof may be one or more. There are a variety of ways to form the convex structure 821. For example, the convex structure 821 may be formed when the guide sheath 82 is molded, or may be formed by thermal reflow on the guide sheath 82. Referring to FIGS. 13B and 13C, the sheath jacket 12 is folded inward as it is withdrawn from the guide sheath 82. Since the outer diameter of the rod portion 11 is smaller than the inner diameter of the guide sheath 82, referring to FIG. 13A, the rod portion 11 is not folded when withdrawn proximally from the guide sheath 82.

In order to facilitate visualization of the position of the sheath jacket 12 in an image, the sheath jacket 12 is provided with a first circumferentially extending marking band 121. Furthermore, in order to facilitate the formation of an inwardly folded crease in the sheath jacket 12, the sheath jacket 12 is provided with a first marking band 121 that extends circumferentially and is not continuous. In some embodiments, the first marking band 121 that extends circumferentially and is not continuous may be formed from a plurality of discrete marking bands. A plurality of discrete marking bands may form a closed first marking band 121 (having no gap), or may form a non-closed first marking band 121 (having at least one gap). In some other embodiments, the first marking band 121 that extends circumferentially and is not continuous may be formed from a segment of marking band that is broken end to end. A segment of marking band that is broken end to end may form a closed first marking band 121 (having no gap), or may form a non-closed first marking band 121 (having at least one gap). Wherein the first marking band 121 may be provided at any position of the sheath jacket 12. In order to facilitate viewing whether the sheath jacket 12 is fully advanced into the guide sheath 82, it is preferred that the first marking band 121 is disposed at the distal end of the sheath jacket 12. In some embodiment, in order to facilitate the sheath jacket 12 to form a crease that is folded radially inward in the lengthwise direction under the extrusion of the convex structure 821, as a preferred embodiment, the sheath jacket 12 is provided with a first marking band 121 that extends circumferentially and is not closed. As a more preferred embodiment, the non-closed portion of the first marking band 121 is aligned with the convex structure 821.

As a preferred embodiment, as shown in FIG. 13C, the distal end of the outer sheath 1 is provided with a first circumferentially extending marking band 121. In some specific embodiments, the first marking band 121 is disposed at the distal end of the sheath jacket 12, the first marking band 121 is provided with a first notch 122 which is aligned with the convex structure 821 in the circumferential direction. It is advantageous that the convex structure 821 presses against the sheath jacket 12 when the sheath jacket 12 is in contact with the convex structure 821. As a result, the sheath jacket 12 forms a crease that is folded radially inward in the lengthwise direction to reduce the outer circumferential dimension of the sheath jacket 12, such that the outer profile of the sheath jacket 12 is reduced.

In some embodiments, the distal end of the guide sheath 82 is provided with a second circumferentially extending marking band 822 to facilitate visualize the guide sheath 82 using an image system. In a preferred embodiment, the distal end of the guide sheath 82 is provided with a second marking band 822 that extends circumferentially and is not closed. The non-closed portion of the second marking band 822 is aligned with the convex structure 821. Wherein, the second non-closed marking band 822 may be formed from a plurality of discrete marking bands, or from a segment of marking band that is broken end to end. In some other embodiments, as shown in FIG. 11, the distal end of the guide sheath 82 is provided with a second circumferentially extending marking band 822 which is provided with a second notch 823. The second marking band 822 is a notched ring, and the convex structure 821 is disposed at the second notch 823, this facilitates the thermal reflow of the guide sheath 82 at the second notch 823 to form the convex structure 821.

In some embodiments, the delivery system further comprises a cardiovascular implant located at the distal end of the outer sheath 1. For example, the cardiovascular prosthetic implant is located within the sheath jacket 12 at the distal end of the outer sheath 1.

In order to facilitate the passage of the sheath jacket 12 through the guide sheath 82, a further modification is made to the sheath jacket 12. In some embodiments, the wall thickness of the sheath jacket 12 is about 0.2 mm, the thickness of the sheath jacket 12 is suitably in the range of 0.18 to 0.22 mm, to provide both reliable support for the implant 23 and case of folding of the sheath jacket 12 as the sheath jacket 12 passes through the guide sheath 82. The sheath jacket 12 can be folded inwardly when being pressed by the convex structure 821, thereby reducing the outer profile of the sheath jacket 12. In some embodiments, the material of the sheath jacket is Nylon or Pebax (Polyether block amide).

The introducer 80 is adapted to guide a cardiovascular implant delivery catheter, which may be an outer sheath 1. As shown in FIG. 11, the introducer 80 includes an introducer handle 81 and a flushing port 811 that is in communication with the guide sheath 82.

The introducer 80, as shown in FIG. 11, is adapted to introduce a cardiovascular implant delivery catheter. The introducer 80 comprises a guide sheath 82, an outer diameter of a distal end of the cardiovascular implant delivery catheter is larger than an inner diameter of the guide sheath 82, and an inner wall of the distal end of the guide sheath is provided with a convex structure; wherein the convex structure reduces an outer profile of the distal end of the cardiovascular implant delivery catheter as the cardiovascular implant delivery catheter enters the guide sheath 82 from the distal end of the guide sheath 82. The outer sheath 1 is a specific embodiment of the cardiovascular implant delivery catheter, and the distal end of the outer sheath 1 is embodied as a sheath jacket 12. When it is desired to withdraw the outer sheath 1 from the guide sheath 82 during a surgical procedure, a convex structure of an inner wall of the distal end of the guide sheath 82 causes the outer profile of the sheath jacket 12 to be reduced to facilitate the withdrawal.

As a preferred embodiment, the distal end of the guide sheath 82 is provided with a second circumferentially extending marking band 822 to facilitate to visualize the guide sheath 82 using an image system. The marking band is provided with a second notch, and the convex structure is arranged at the second notch. The second marking band 822 is a notched ring, and the convex structure 821 is disposed at the second notch 823, this facilitates the thermal reflow of the guide sheath 82 at the second notch 823 to form the convex structure 821.

### The Fourth Embodiment

Referring to FIGS. 8A to 8C, an implant delivery system is adapted to deliver a cardiovascular prosthetic implant, and comprises an outer sheath 1, an inner sheath 21, a hemostatic seal assembly and a plurality of first movable elongate members 2. The inner sheath 21 extends through and is disposed in the outer sheath 1, and the proximal end of the inner sheath 21 extends beyond the proximal end of the outer sheath 1. The plurality of first movable elongate members 2 extend through the inner sheath 21, and the proximal ends of the plurality of first movable elongate members 2 extend beyond the proximal end of the inner sheath 21. The hemostatic seal assembly comprises a first seal member and a second seal member. The first scal member is configured to seal the gap between the outer sheath 1 and the inner sheath 21, and the second seal member is configured to seal the gap between the inner sheath 21 and the plurality of first movable elongate members 2. The inner sheath 21 provides an independent space for the first movable elongate member 2, avoiding interference to the first movable elongate member by other members within the outer sheath 1. The first movable elongate member 2 is movable relative to the inner sheath 21 and the second seal member. By means of the provision of the second seal member, it is ensured that the fluid in the inner sheath 21 does not flow out during the movement of the first movable elongate member 2.

In some embodiments, the outer sheath 1, the inner sheath 21, and the hemostatic seal assembly are fixedly connected. In some other embodiments, the second seal member may also be detachably connected to the inner sheath 21.

Specifically, referring to FIGS. 8B, 8C and 9B, the first seal member includes a scaling gasket 63 which is provided with a first through hole 661. The inner sheath 21 passes through and is sealed by the first through hole 661. The second seal member is configured as a necking member 22 which is configured to be adapted to provide a longitudinally movable sealing for the plurality of first movable elongate members 2 relative to the inner sheath 21. As shown in FIGS. 8B and 8C, the necking member 22 is connected to the end of the inner sheath 21, and the first movable elongate member 2 extends out via the necking member 22. Specifically, as shown in FIGS. 9A and 9B, the necking member 22 includes a necking connection portion 221 and a necking seal portion 222 connected to the proximal end of the necking connection portion 221. The necking seal portion 222 provides a longitudinally movable scaling for the plurality of first movable elongate members 2 relative to the inner sheath 21.

Referring to FIGS. 8A to 9B, in some embodiments, the delivery system further comprises a carriage 6 which is provided with a carriage cavity 611 that is in communication with the lumen of the outer sheath 1. The proximal end of the outer sheath 1, the inner sheath 21 and the sealing gasket 63 are all fixed to the carriage 6; wherein the carriage 6 is configured to drive the outer sheath 1 and the inner sheath 21 to move longitudinally relative to the plurality of first movable elongate members 21. The cardiovascular implant mounted at the distal end of the first movable elongate member 21 can be released by the carriage 6 driving the outer sheath 1 and the inner sheath 21 to move longitudinally relative to the plurality of first movable elongate members 2.

Continuing to refer to FIGS. 8A to 9B, in some embodiments, the delivery system further comprises a second movable elongate member which extends through and is disposed in the outer sheath 1, and a proximal end of which extends beyond the proximal end of the outer sheath 1. The sealing gasket 63 is further provided with a second through hole 662 through which the second movable elongate member passes and by which the sealing is provided. The carriage 6 is configured to further drive the outer sheath 1 and the inner sheath 21 to move longitudinally relative to the second movable elongate member. The second movable elongate member may be a guidewire tube 4 and/or a balloon tube 3. The outer sheath 1 and the inner sheath 21 can move longitudinally relative to the second movable elongate member. The first movable elongate member 2 is located inside the inner sheath 21, and the second movable elongate member is located outside the inner sheath 21. The inner sheath 21 separates the first movable elongate member 2 from the second movable elongate member to avoid interference therebetween. The sealing gasket 63 may be fixedly connected to the outer sheath 1. The second movable elongate member extends out via the sealing gasket 63 by which the second movable elongate member is scaled.

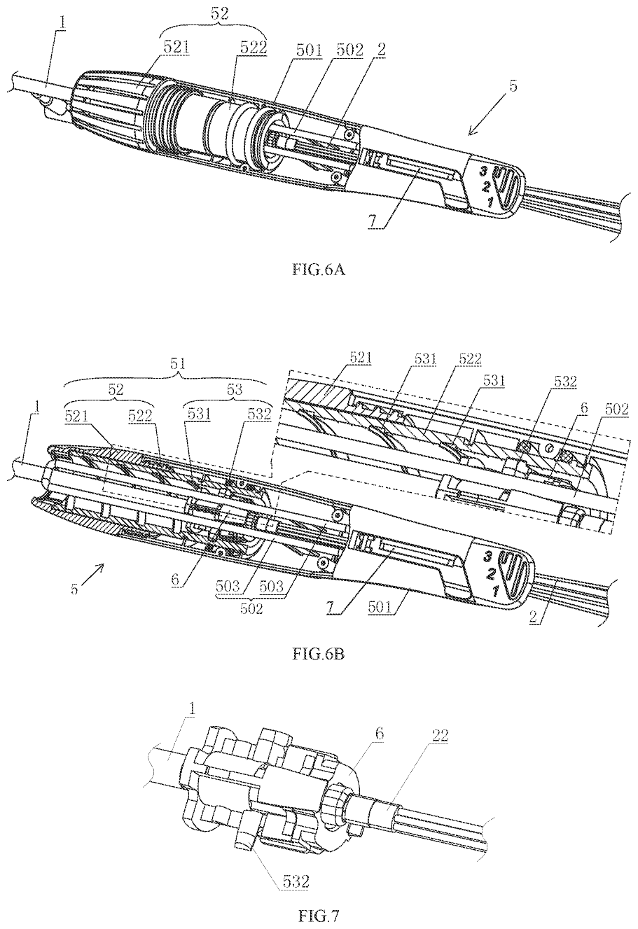

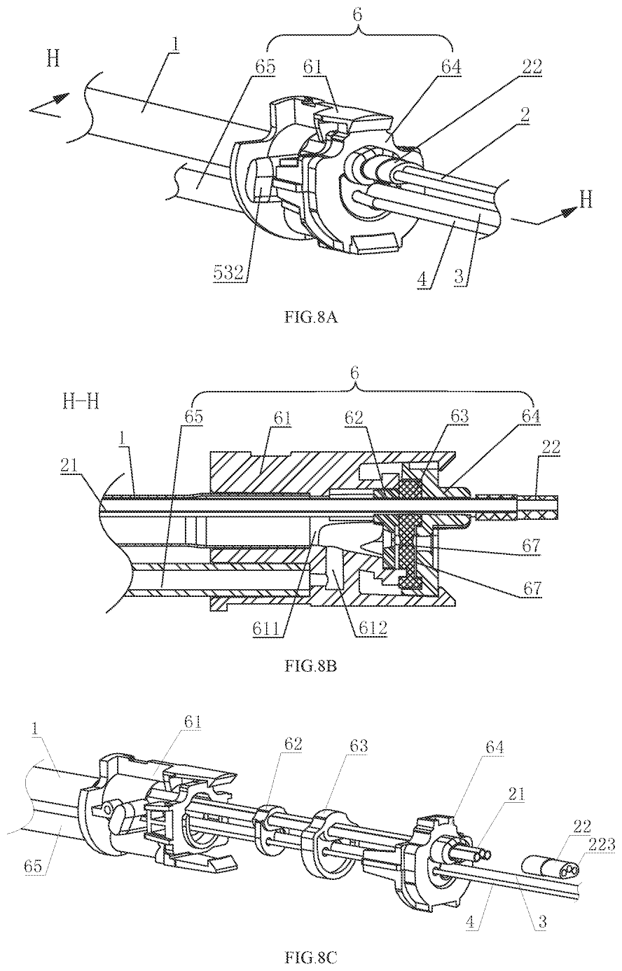

In some specific embodiments, as shown in FIGS. 6A to 7, the delivery system further comprises a handle 5 by which the sheath jacket 12 is driven to move proximally, so as to release the implant 23. The handle 5 includes the carriage 6 and a driving mechanism 51. The driving mechanism 51 is coupled to the carriage 6. The proximal end of the rod portion 11 of the outer sheath 1 is fixedly connected to the carriage 6. The driving mechanism 51 is configured to drive the carriage 6 to move longitudinally. The driving mechanism 51 may drive the carriage 6 to move proximally in the longitudinal direction. The carriage 6 drives the outer sheath 1 to move together proximally, so that the implant 23 is released from the sheath jacket 12 of the outer sheath 1. The inner sheath is embodied as a three-lumen tube 21 which can be mounted on the carriage 6. The carriage 6 is configured to drive the outer sheath 1 and the three-lumen tube 21 to move longitudinally. The proximal end of the three-lumen tube 21 is fixedly connected to the carriage 6, and the three-lumen tube 21 moves together with the outer sheath 1 as the carriage 6 moves. As shown in FIGS. 8A and 8C, the second movable elongate member extends through the carriage 6 and is capable of longitudinal movement relative to the carriage 6. In some specific embodiments, as shown in FIGS. 6A and 6B, the handle 5 includes a handle housing 501 to which a carriage 6 is movably mounted and which is restrained from rotating relative to the handle housing 501. The driving mechanism 51 comprises a rotating part 52 which is connected to the carriage 6 by means of a spiral structure 53. The spiral structure 53 is configured to bring about a translatory movement of the carriage 6 in the handle housing 501 when the rotating part 52 makes rotation movement. Since the carriage 6 is restrained from rotating relative to the handle housing 501, when the rotating part 52 rotates relative to the handle housing 501, the rotating part 52 drives the carriage 6 to move in the longitudinal direction on the handle housing 501 through the spiral structure 53. Specifically, as shown in FIGS. 6B and 7, the spiral structure 53 includes a spiral groove 531 provided on the rotating part 52 and a bump 532 disposed on the carriage 6, and the bump 532 is slidably embedded in the spiral groove 531. A guide rod 502 extending in the longitudinal direction is fixedly arranged in the handle housing 501, and the carriage 6 cooperates with the guide rod 502 to prevent the carriage 6 from rotating and to guide the longitudinal movement of the carriage 6. Specifically, as shown in FIG. 6B, the guide rod 502 includes two guide rods 503 which are parallel to each other and extend in the longitudinal direction. The two guide rods 503 are respectively located on both sides of the carriage 6 and are in contact and matching with the side wall of the carriage 6. The carriage 6 is restrained between the two guide rods 503, and the carriage 6 is restrained from rotating, and is movable along the guide rods 503. The rotating part 52 is capable of rotational movement relative to the handle housing 501 and is restrained from longitudinal movement relative to the handle housing 501. The rotating part 52 includes a knob 521 and a screw part 522. The screw part 522 has a cylindrical shape. The spiral groove 531 is disposed on the inner wall of the screw part 522. The knob 521 is fixedly connected to the screw part 522, and at least partially extends out of the handle housing 501, so as to facilitate the hand-holding operation.

As shown in FIGS. 8A to 9B, a detailed description is given below by taking an example that the second movable elongate member includes the guidewire tube 4 and the balloon tube 3. The guidewire tube 4, the balloon tube 3, and the positioning wire 2 pass through the carriage 6 and out from the proximal end of the handle 5. The sealing gasket 63 is provided in the carriage 6. The carriage 6 includes a carriage body 61, a carriage plate 62 and a carriage cover 64. The proximal end of the outer sheath 1 is fixedly connected to the carriage body 61, and a carriage cavity 611 is disposed inside the carriage body 61. The carriage cover 64, the sealing gasket 63, and the carriage plate 62 are all mounted on the proximal side of the carriage body 611 and are sequentially distributed in a direction from the proximal end to the distal end. The carriage plate 62 and the carriage cover 64 are configured to fix the sealing gasket 63 to the carriage body 61. The carriage cover 64, the sealing gasket 63, and the carriage plate 62 are provided with a first through hole 661, a second through hole 662, and a third through hole 663, respectively. The positioning wire 2 disposed to pass through the inner sheath 21 within the outer sheath 1 extends beyond the carriage 6 via the carriage cavity 611 and the first through hole 661. The balloon tube 3 disposed to pass through the outer sheath 1 extends beyond the carriage 6 via the carriage cavity 611 and the second through hole 662. The guidewire tube 4 disposed to pass through the outer sheath 1 extends beyond the carriage 6 via the carriage cavity 611 and the third through hole 663. Specifically, as shown in FIGS. 8C and 9B, the positioning wire 2 (not shown) is disposed to pass through the three-lumen tube 21. The three-lumen tube 21 extends beyond the carriage 6 via the carriage cavity 611 and all the first through holes 661. The proximal end of the three-lumen tube 21 is fixedly connected to the first through hole 661 of the carriage cover 64 and extends out of the first through hole 661. The three-lumen tube 21, the guidewire tube 4 and balloon tube 3 extend through the carriage 6 after extending out of the proximal end of the outer sheath 1. The three-lumen tube 21, the guidewire tube 4 and the balloon tube 3 passing through the carriage 6 are sealed by a sealing gasket 63, to prevent blood inside the outer sheath 1 from leaking outside along the outer wall of the three-lumen tube 21, the outer wall of the guidewire tube 4, or the outer wall of the balloon tube 3.

In some embodiments, the inner sheath 21 and the second movable elongate member extend longitudinally through the carriage plate 62 and the carriage cover 64, respectively; wherein corresponding to the longitudinally extending position of the second movable elongate member, a gap 67 is provided between the sealing gasket 63 and the carriage cover 64, and between the sealing gasket 63 and carriage plate 62, respectively. Referring to FIGS. 8B and 8C, the gap 67 is provided around the balloon tube 3 and the guidewire tube 4. The gap 67 can provide a deformation space for the sealing gasket 63 when the balloon tube 3 and the guidewire tube 4 pass through the sealing gasket 63, so as to reduce the friction force during the movement of the balloon tube 3 or the guidewire tube 4 relative to the sealing gasket 63, and facilitate the movement of the balloon tube 3 and/or the guidewire tube 4. Specifically, as shown in FIGS. 8C to 9B, the gap 67 may be formed between the sealing gasket 63 and the carriage plate 62 by providing a groove at the distal end surface of the sealing gasket 63 or providing a groove at the proximal end surface of the carriage plate 62. The gap 67 may be formed between the sealing gasket 63 and the carriage cover 64 by providing a groove at the proximal end surface of the sealing gasket 63 or providing a groove at the distal end surface of the carriage cover 64.

In some specific embodiments, the first movable elongate member 2 is a positioning wire 2, and the number of the positioning wire 2 may be three. The inner sheath 21 may be a three-lumen tube 21. The three-lumen tube 21 includes three independent lumens to be adapted to accommodate three positioning wires. The three positioning wires 2 are located respectively in independent lumens, which can avoid mutual contact between the three positioning wires and mutual interference during the movement of the positioning wires 2. The necking connection portion 221 has a connection cavity whose cross-sectional shape is configured to conform to the shape of the outer profile of the inner sheath. The necking seal portion 222 has three sealing cavities that provide sealing for three positioning wires. As shown in FIGS. 8B, 9A and 9B, the inner sheath 21 is a three-lumen tube, the proximal end of which is connected with a necking member 22. The necking member 22 includes a necking connection portion 221 and a necking seal portion 222. The three-lumen tube 21 is connected to the necking connection portion 221, and the necking seal portion 222 is provided with three positioning wire holes 223 via which the proximal end of the positioning wire 2 extends beyond the necking member 22. The cavity of the necking connection portion 221 is in communication with the three-lumen tube 21, and the positioning wire hole 223 is in communication with the cavity of the necking connection portion 221, and each positioning wire 2 is sealed by the positioning wire hole 223, so that the sealing effect on the positioning wire 2 is improved. The number of the positioning wire hole 223 provided in the three-lumen tube 21 can be set according to the number of the positioning wire 2, so that the positioning wires 2 and the positioning wire holes 223 correspond to each other one by one. In a preferred embodiment, the delivery system includes three positioning wires 2, and correspondingly, three positioning wire holes 223 are arranged on the three-lumen tube 21. The second movable elongate member may be a guidewire tube 4 and/or a balloon tube 3. The inner sheath may be made of PEEK and the necking member is made of PEBAX (Polyether block amide). PEBAX has a number of excellent properties between thermoplastic elastomers and rubber bodies and is easy to process.

As shown in FIGS. 8A and 8B, the carriage body 61 is connected with a flush port 65, which communicates with the carriage cavity 611 and with the outer sheath 1. The flush port 65 can be used to deliver a flushing material to the carriage 6 and outer sheath 1. Specifically, the carriage body 61 is provided with a flushing hole 612 to which the flush port 65 is connected. The flushing hole 612 is in communication with the carriage cavity 611 which is in communication with the outer sheath 1, so as to achieve that the flush port 65 communicates both with the carriage cavity 611 and with the outer sheath 1. The flush port 65 may be coupled to the carriage body 61 such that movement of the carriage body 61 can cause movement of the flush port 65.

In an embodiment, the carriage body 61, the carriage plate 62 and the carriage cover 64 are made of an injection molded plastic, such as polycarbonate, phenolic resin molding plastics, or the like. The carriage body 61, the carriage plate 62, and the carriage cover 64 may be made of transparent materials, preferably, to allow a UV (Ultra-Violet Ray) bonding process. The carriage body 61 and the flush port 65 are connected by a UV bonding process, and the carriage cover 64 and the three-lumen tube 21 are connected also by the UV bonding process. Specifically, the contact surfaces of the carriage body 61 and the flush port 65 are coated with UV glue, respectively. The UV glue is irradiated by UV light and is cured to secure the carriage body 61 and the flush port 65 together. The carriage body 61 and the carriage plate 62 may be separately molded and connected together, or may be integrally molded. The sealing gasket 63 may be composed of an elastic polymer, which may be silicone, polyurethane, latex; the elastic polymer is preferably nylon.

FIGS. 10A to 10H show a partial schematic view of the distal end of the delivery system in a surgical procedure. Referring to FIG. 10A, the distal end of the outer sheath 1 is advanced along the vessel to an appropriate location in the body (e.g., proximate to the implantation position). Referring to FIG. 10B, the guidewire tube 4 and the nose cone 41 move distally, and the nose cone 41 is separated from the outer sheath 1. Referring to FIG. 10C, by rotating the rotating part 52 on the handle 5, the carriage 6 drives the sheath jacket 12 to move together proximally to release the implant 23. Referring to FIG. 10D, the implant 23 is inflated and adjusted to the diseased physiological valve by filling the implant 23 with the filler through the positioning wire 2. Referring to FIG. 10E, the balloon tube 3 moves distally, and the balloon 34 moves distally under the guidance of the guidewire tube 4 into the implant 23. Referring to FIG. 10F, the balloon 34 is filled with the filler through the balloon tube 3, and the inflated implant 23 is expanded and reshaped after the balloon 34 is inflated. Referring to FIG. 10G, after the balloon 34 has been contracted, it is driven by the balloon tube 3 to move together proximally, and it is withdrawn from the implant 23. Referring to FIG. 10H, the positioning wire 2 is detached from the implant 23, and the delivery system is withdrawn from the human body.

As shown in FIGS. 1A, 1B and 11, the delivery system incorporates a balloon assembly. The expanded or inflated implant 23 (such as heart valve prosthesis) can be expanded and reshaped by the balloon 34, facilitating the implanted valve to meet clinical requirements in terms of expansion and differential pressure. During surgery, the use of the integrated balloon 34 to expand and reshape the expanded heart valve prosthesis can effectively simplify the surgical process, and shorten the surgical operation time. The nose cone 41 in this delivery system is provided with side holes 43. In the case where the shaft hole 42 is blocked, the side holes 43 keep the pressure of blood flow in the periphery of the nose cone 41 consistent with the pressure of blood flow in the cavity of the nose cone 41, and this facilitates accurate measurement of the blood flow pressure at the periphery of the nose cone 41 by a pressure sensor disposed within the nose cone 41. In the handle 5 of the delivery system, the carriage 6 seals the positioning wire 2, the guidewire tube 4 and the balloon tube 3, and has a good sealing effect. The guide sheath 82 in this delivery system is provided with a convex structure 821 that allows the sheath jacket 12 to be folded longitudinally inward as it is withdrawn proximally from the guide sheath 82 to reduce the outer peripheral dimension of the sheath jacket 12, so as to facilitate the smooth withdrawal of the sheath jacket 12.

### The Fifth Embodiment

As shown in FIGS. 1A and 4A to 5, a blood flow pressure monitoring system comprises a guidewire tube 4, a pressure sensor, and a nose cone 41 connected to a distal end of the guidewire tube 4. The nose cone 41 is provided with a shaft hole 42 that is in communication with the guidewire tube 4 and a plurality of side holes that are in communication with the shaft hole 42. The pressure sensor is configured to sense pressure of blood flowing through at least one hole of the plurality of side holes and the shaft hole. The shaft hole 42 is configured to allow a guidewire in the guidewire tube 4 to pass therethrough. A pressure sensor (not shown) may be provided in the shaft hole or side holes of the nose cone 41 or in the guidewire tube 4. When the end of the shaft hole 42 is blocked by the heart wall, the pressure of blood flow in the periphery of the nose cone 41 can be kept consistent with the pressure of blood flow in the nose cone 41 by the plurality of side holes 43, and this facilitates accurate measurement of the blood flow pressure at the periphery of the nose cone 41 by the pressure sensor disposed within the nose cone 41 or in the guidewire tube 4. In some embodiments, the blood flow pressure monitoring system is a blood flow pressure monitoring system suitable for deployment of a cardiovascular implant. The blood flow pressure monitoring system comprises an implant delivery system, having the guidewire tube 4 and the nose cone 41 as components thereof. The specific structure and operating mode of the guidewire tube 4, the nose cone 41 and the implant delivery system have been described in detail in the foregoing, and will not be described again.

The pressure sensor is used for sensing blood flow pressure, and may be placed in the nose cone 41 or in the guidewire tube 4. The center line of the side hole 43 may or may not be perpendicular to the axis of the nose cone 41. As shown in FIG. 4B, the nose cone 41 is laterally symmetrically provided with two pairs of side holes 43, which are symmetrical with respect to the plane of the axis over the nose cone.

In some embodiments, the implant delivery system further comprises an outer sheath 1, and the nose cone 41 is configured to be detachably connected to the outer sheath 1. The nose cone 41 has a connection segment 45 connected to the distal end of the outer sheath 1. The connection segment 45 at the proximal end of the nose cone 41 is detachably connected to the distal end of the outer sheath 1. In a preferred embodiment, the outer diameter of the connection segment 45 of the nose cone 41 is in an interference fit with the inner diameter at the distal end of the outer sheath 1. This can effectively reduce a fish mouth formed by the sheath jacket 12 when the nose cone 41 is bent.

In some embodiments, the outer side wall of the nose cone 41 is provided with a flushing groove 44 in communication with the inner cavity of the outer sheath 1. During the flushing of the inner cavity of the outer sheath 1, air and liquid can be smoothly discharged from the flushing groove 44. Specifically, the flushing groove 44 extends longitudinally through the outer side wall of the nose cone 41 along the proximal end portion of the nose cone 41. A plurality of flushing grooves 44 are distributed along the circumferential direction of the nose cone 41 on the side wall of the nose cone 41.

In this embodiment, the implant delivery system may include an outer sheath, and the nose cone is configured to be detachably coupled to the outer sheath.

In an embodiment, the cardiovascular implant is a cardiac aortic valve prosthesis, and the pressure sensor is configured to sense blood flow pressure in the left ventricle.

In an embodiment, the blood flow pressure monitoring system is adapted for transcatheter aortic valve replacement to assist in monitoring blood flow pressure in the left ventricle.

The present disclosure further provides a device for assisting in monitoring blood flow pressure, suitable for transcatheter heart valve replacement or repair, the device including a guidewire tube 4 and a nose cone 41 connected to a distal end of the guidewire tube 4. The nose cone 41 is provided with a shaft hole 42 that is in communication with the guidewire tube 4 and a plurality of side holes 43. At the time of detecting the blood flow pressure, a pressure sensor for detecting the blood flow pressure in the periphery of the nose cone 41 is disposed in the nose cone 41 or the guidewire tube 4. The arrangement position of the pressure sensor, the arrangement mode of the side holes 43 and the effect thereof have been described in detail in the foregoing, and are not described here again.

In some embodiments, the device for assisting in monitoring blood flow pressure is adapted for transcatheter aortic valve replacement via the positioning wire to assist in monitoring blood flow pressure in the left ventricle.

In the blood flow pressure monitoring system or the device for assisting in monitoring blood flow pressure provided by the present disclosure, the nose cone is provided with both of a shaft hole and side holes, such that when part of the holes are blocked by the tissue wall, at least one hole can be open to the actual blood flow environment, so that the sensor can sense the actual hemodynamic parameter value, and the accuracy of the monitoring data can be improved. In addition, due to the presence of the shaft hole and several side holes, each time the monitored value of the sensor can basically reflect the actual blood flow situation, so that the number of times that the doctor repeatedly adjusts and repeats the measurement in pursuit of the monitoring accuracy can be reduced, thereby improving the efficiency of surgery.

### The Sixth Embodiment

Some embodiments are provided that specify a method of operating an implant delivery system.

The method comprises: before using the delivery system, connecting the positioning wire 2 to the implant 23; loading the implant 23 into the sheath jacket 12 by a loading tool after folding the implant 23; flushing the guidewire tube 4 with physiological saline; pulling the guidewire tube 4 to connect the nose cone 41 to the sheath jacket 12; introducing saline from the flush port 65 of the carriage 6 in the handle 5 to flush the outer sheath 1.

In some embodiments, the implant 23 is deployed to a diseased valve position, for example, to an aortic position, by minimally invasive surgery using the prosthetic delivery system described above. In some embodiments, the method generally comprises entering the aorta through the femoral artery. The vascular access site may be prepared according to standard practice, and the guidewire may be inserted into the left ventricle through a vascular access.

Referring to FIG. 10A, a delivery system in which a cardiovascular prosthetic implant 23 is placed may be advanced through a blood vessel. In some embodiments, a guidewire (not shown) is inserted into the delivery system. In such an embodiment, the nose cone 41 may enter the blood vessel directly via the guidewire, and the nose cone 41 can expand the blood vessel to facilitate smooth advancement of the outer sheath 1 in the vessel. The outer sheath 1 can be advanced to a position close to the diseased valve.

Referring to FIGS. 10B and 10C, in some embodiments, the implant 23 can be exposed or released in place (for example, at or below the diseased valve) by partially or fully retracting the outer sheath 1 while the plurality of positioning wires 2 are held stationary. At this point of time, the balloon 34 remains in the outer sheath 1 or partially exposed from the outer sheath 1. In some embodiments, the implant 23 may also be exposed or released by pushing the positioning wire 2 distally while the outer sheath 1 is held stationary, with the balloon 34 remaining in place in the outer sheath 1. Referring to FIG. 10D, once the implant 23 is released from the outer sheath 1, it can move proximally or distally, and a fluid or inflating medium may be introduced into the cuff of the implant 23 to provide shape and structural integrity. The positioning wire 2 can be used both to transport medium to fill the cuff of the implant 23 and to control the implant 23 to be positioned at the implantation position.

Referring to FIG. 10E, in some embodiments, the balloon 34 integrated into the delivery system is advanced into alignment with the implant 23 under the guidance of the guidewire tube 4, that is, the middle portion of the balloon 34 is located in the implant 23. Referring to FIG. 10F, the balloon 34 may be inflated to further expand the implant 23, which may promote sufficient inflation of the implant 23 and achieve good adhesion to the physiological structure. The balloon 34 with a pressure of 1˜3 ATMs will help to re-expand the inflated prosthesis. In some embodiments, as the balloon 34 expands the implant 23, the pressure within the cuff of the implant 23 is rapidly increased to 10˜12 ATMs, or to a higher pressure value.

In some embodiments, the balloon 34 integrated on the delivery system may be used to expand the diseased tissue valve before the implant 23 is positioned at the implantation position. The balloon 34 expands the diseased tissue valve in a manner similar to the manner in which it expands the implant 23 and will not be described again.

Referring to FIG. 11, in some embodiments, in a prosthesis delivery system incorporating an introducer 80, the guide sheath 82 may be pre-installed along the outer sheath 1 prior to performing the minimally invasive surgery. For example, the manufacturer may pre-install the guide sheath 82 along the outer sheath 1. For example, in some arrangements, it may be appropriate to extend the proximal end of the outer sheath 1 through the guide sheath 82 before attaching the handle to the proximal end of the outer sheath 1.

Referring to FIGS. 13A to 13C, in some embodiments, when the outer sheath 12 at the distal end of the outer sheath 1 is withdrawn proximally from the guide sheath 82, the sheath jacket 12 forms a crease that is folded radially inward in the lengthwise direction via the convex structure 821 of the guide sheath 82 to reduce the outer circumferential dimension of the sheath jacket 12, so as to allow the sheath jacket 12 to enter the guide sheath 82.

The foregoings are only several embodiments of the present disclosure, and those skilled in the art may make various modifications or variations to the embodiments of the present disclosure according to the disclosure of the application documents without departing from the spirit and scope of the present disclosure.

1. An implant delivery system adapted to deliver a cardiovascular prosthetic implant, characterized in comprising:
an outer sheath;
a balloon and a balloon tube, the balloon being connected to a distal end of the balloon tube; and
a nose cone and a guidewire tube, the nose cone being connected to a distal end of the guidewire tube;
wherein the balloon tube and the guidewire tube are disposed within the outer sheath in a longitudinal extension manner and are configured to be operable to pass through a distal end of the outer sheath.

2. The delivery system according to claim 1, characterized in that, the guidewire tube is disposed to partially extend through the balloon tube, and the guidewire tube includes a free segment and a bound segment adjacent to the free segment,
the bound segment is defined by a length of the guidewire tube extending longitudinally through the balloon tube,
the free segment is defined by a length of the guidewire tube located outside of the balloon tube and extending longitudinally in parallel with the balloon tube.

3. The delivery system according to claim 2, characterized in that, the guidewire tube is configured to be longitudinally movable relative to the balloon tube.

4. The delivery system according to claim 3, characterized in that, the balloon tube includes a proximal segment tube, a middle segment tube and a distal segment tube, the middle segment tube is located between the proximal segment tube and the distal segment tube, and the bound segment of the guidewire tube extends longitudinally through the distal segment tube; wherein the outer diameter of the middle segment tube is smaller than that of the proximal segment tube.

5. The delivery system according to claim 4, characterized in that, a side wall opening is provided near the proximal end of the distal segment tube, and the bound segment of the guidewire tube extends longitudinally through the distal segment tube via the side wall opening.

6. The delivery system according to claim 5, characterized in that, the distal segment tube is a double-lumen tube, the double-lumen tube has a first lumen communicating with the balloon and a second lumen isolated from the balloon, the first lumen is in communication with the lumen of the middle segment tube; and the bound segment of the guidewire tube extends longitudinally through the second lumen.

7. The delivery system according to claim 6, characterized in that, an inner tube isolated from the inner cavity of the balloon is arranged inside the balloon, the second lumen is in communication with the inner tube from the proximal end of the inner tube; and the bound segment of the guidewire tube extends longitudinally through the inner tube.

8. The delivery system according to claim 6, characterized in that, the cavities of the middle segment tube and the proximal segment tube are circular in cross section, and the cross section of the cavity of the first lumen of the distal segment tube is quasi crescent-shaped.

9. The delivery system according to claim 2, characterized in that, the delivery system further comprises a plurality of positioning wires and an inner sheath adapted to accommodate the plurality of the positioning wires, the plurality of the positioning wires being configured such that distal ends thereof are connected to an implant; wherein a proximal end of the inner sheath, a proximal ends of the guidewire tube and a proximal end of the balloon tube are configured to extend longitudinally through the outer sheath in parallel and laterally non-nested with each other.

10. The delivery system according to claim 9, characterized in that, the delivery system further comprises a handle to which the proximal end of the outer sheath is connected; the handle is configured to drive the outer sheath to move proximally to release the implant.

11. An implant delivery system, characterized in comprising:
an outer sheath;
a plurality of positioning wires and an inner sheath adapted to accommodate the plurality of the positioning wires, the plurality of the positioning wires being configured such that distal ends thereof are connected to an implant;
a balloon and a balloon tube, the balloon being connected to a distal end of the balloon tube; and
a nose cone and a guidewire tube, the nose cone being connected to a distal end of the guidewire tube;
wherein the inner sheath, the guidewire tube and the balloon tube run through and are disposed in the outer sheath, and the body portions of the inner sheath, the guidewire tube, and the balloon tube are configured to extend longitudinally through the outer sheath in parallel and laterally non-nested with each other.

12. The implant delivery system according to claim 11, characterized in that, the inner sheath is separate from the outer sheath, and the material of the inner sheath has a stiffness greater than that of the outer sheath.

13. The implant delivery system according to claim 12, characterized in that, the inner sheath is a three-lumen tube, and the three-lumen tube includes three independent lumens to be adapted to accommodate three positioning wires.

14. The implant delivery system according to claim 13, characterized in that, an outer profile of the inner sheath is arranged close to the inner peripheral wall of the outer sheath.

15. The implant delivery system according to claim 13, characterized in that, the three lumens of the inner sheath are distributed in an arc shape, and the diameter of an circumcircle of the outer profile of the inner sheath is equal to the inner diameter of the outer sheath.

16. The implant delivery system according to claim 12, characterized in that, the material of the outer sheath is Nylon, and the material of the inner sheath is PEEK.

17. The implant delivery system according to claim 11, characterized in that, the delivery system further comprises a handle,
the proximal end of the outer sheath and the proximal end of the inner sheath are connected to the handle;
the proximal end of the balloon tube, the proximal end of the guidewire tube and the proximal ends of the plurality of positioning wires all extend beyond the handle;
the handle is provided with a first seal member which is configured to provide sealing for the inner sheath, the balloon tube and the guidewire tube;
wherein the sealing provided by the first seal member for the balloon tube and the guidewire tube is a longitudinally movable sealing.

18. The implant delivery system according to claim 17, characterized in that, the handle is further provided therein with a second seal member which is configured to provide a longitudinally movable sealing for the plurality of positioning wires.

19. The implant delivery system according to claim 18, characterized in that, the first seal member is configured as a sealing gasket, and the second seal member is configured as a necking member.
